(19) United States
(12) Patent Application Publication
(10) Pub. No.: US 2023/0203452 A1
(43) Pub. Date: Jun. 29, 2023

(54) METHOD OF PRODUCING CONDITIONED MEDIUM FOR CULTURING PATIENT-DERIVED CANCER CELLS

(71) Applicant: TOPPAN Inc., Taito-ku (JP)

(72) Inventors: Kei TSUKAMOTO, Taito-ku (JP); Shiro KITANO, Taito-ku (JP); Tomoko KUNITOMI, Taito-ku (JP)

(73) Assignee: TOPPAN Inc., Taito-ku (JP)

(21) Appl. No.: 18/171,832

(22) Filed: Feb. 21, 2023

Related U.S. Application Data
(63) Continuation of application No. PCT/JP2021/030361, filed on Aug. 19, 2021.

(30) Foreign Application Priority Data
Aug. 21, 2020 (JP) 2020-140076

(51) Int. Cl. C12N 5/09

(52) U.S. Cl. CPC C12N 5/0693; C12N 2502/28; C12N 2502/1323; C12N 2513/00; C12N 2533/90

(57) ABSTRACT

A method of producing a conditioned medium for culturing a patient-derived cancer cell, including culturing, in a first medium for 24 hours or longer, a three-dimensional cell tissue including an extracellular matrix component, a polymer electrolyte, and a cell cluster including a fibroblast, and collecting the first medium in which the three-dimensional cell tissue has been cultured as the conditioned medium.

### CROSS-REFERENCE TO RELATED APPLICATIONS

The present application is a continuation of International Application No. PCT/JP2021/030361, filed Aug. 19, 2021, which is based upon and claims the benefits of priority to Japanese Application No. 2020-140076, filed Aug. 21, 2020. The entire contents of all of the above applications are incorporated herein by reference.

### BACKGROUND OF THE INVENTION

### Field of the Invention

The present invention generally relates to a method of producing a conditioned medium for culturing a patient-derived cancer cell. More specifically, the present invention relates to a method of producing a conditioned medium for culturing a patient-derived cancer cell, a conditioned medium for culturing a patient-derived cancer cell, and a method for culturing a patient-derived cancer cell.

### Discussion of the Background

Culturing cells taken from living organisms can be difficult. Cells taken from living organisms may sometimes be cultured by using a special medium, but such media are expensive and may be difficult to use. Therefore, there is a need for a technique to culture cells collected from living organisms more cost-effectively and simply.

The inventors of the present invention have previously developed a technique of producing three-dimensional cell tissue, including: a step of obtaining a mixture including cells suspended in a solution containing at least a cationic buffer solution, an extracellular matrix component, and a polymer electrolyte; a step of collecting the cells from the obtained mixture to form a cell aggregate on a substrate; and a step of culturing the cells to obtain three-dimensional cell tissue (see JP 6639634 B).

### Summary of the Invention

According to an aspect of the present invention, a method of producing a conditioned medium for culturing a patient-derived cancer cell includes culturing, in a first medium for 24 hours or longer, a three-dimensional cell tissue including an extracellular matrix component, a polymer electrolyte, and a cell cluster including a fibroblast, and collecting the first medium in which the three-dimensional cell tissue has been cultured as the conditioned medium.

According to another aspect of the present invention, a method of culturing a patient-derived cancer cell includes preparing a conditioned medium, and culturing a patient-derived cancer cell in a second medium including the conditioned medium for culturing a patient-derived cancer cell. The preparing includes culturing, in a first medium for 24 hours or longer, a three-dimensional cell tissue including an extracellular matrix component, a polymer electrolyte, and a cell cluster including a fibroblast, and collecting the first medium in which the three-dimensional cell tissue has been cultured as the conditioned medium.

### DESCRIPTION OF THE EMBODIMENTS

### Method of Producing Conditioned Medium for Culturing Patient-Derived Cancer Cell

According to one embodiment, the present invention provides a method of producing a conditioned medium for culturing a patient-derived cancer cell, the method including: culturing three-dimensional cell tissue in a first medium for 24 hours or longer, the three-dimensional cell tissue containing a cell cluster, an extracellular matrix component, and a polymer electrolyte, the cell cluster containing a fibroblast; and collecting the first medium in which the three-dimensional cell tissue has been cultured for use as the conditioned medium for culturing a patient-derived cancer cell.

The conditioned medium, which is also referred to as acclimation medium, conditioned medium, conditioned culture medium, or aged culture medium, is a medium in which cells are recovered from the culture after culture. The conditioned medium contains various factors secreted by cells.

A patient-derived cancer cell is a primary cell taken from a living organism or a near-primary cell. A near-primary cell refers to a cell that has not been established and has a finite life span. A near-primary cell may be a cell that has a small number of passages and has divided approximately 80 times or less after being collected from a living organism. The patient-derived cancer cell may be a single type of cell or a mixture of multiple types of cells.

As described below in the Examples, the conditioned medium of the present embodiment is suitable for use in culturing patient-derived cancer cells. By using the conditioned medium of the present embodiment, patient-derived cancer cells can be cultured without using a special medium. The term “special medium” as used in the present specification refers to an ES cell culture medium (for example, ES cell culture medium, Gibco), StemPro hESC SFM-Human Embryonic Stem Cell Culture Medium (Thermo Fisher), and the like.

The term “three-dimensional cell tissue” as used in the present specification refers to a three-dimensional cell aggregate. The form of the three-dimensional cell tissue is not particularly limited. For example, it may be three-dimensional cell tissue formed by culturing cells inside a cell culture insert, three-dimensional cell tissue formed by culturing cells inside a scaffold of a natural biopolymer such as collagen or a synthetic polymer, a cell aggregate (also referred to as a spheroid), or a sheet-like cell structure.

The three-dimensional cell structure can be obtained by conducting step (A), step (B), and step (C). In step (A), a mixture is obtained containing a cell cluster, an extracellular matrix component, and a polymer electrolyte, the cell cluster containing a fibroblast. In step (B), a cell aggregate is obtained from the mixture. In step (C), three-dimensional cell tissue is obtained by culturing the cell aggregate. Each step will be described below.

Firstly, in step (A), a mixture is obtained containing a cell cluster, an extracellular matrix component, and a polymer electrolyte, the cell cluster containing a fibroblast. Whether a cell is a fibroblast can be determined by the morphology of the cell as viewed under a microscope or by the expression of marker molecules from the cell. Markers indicating fibroblasts include Fibroblast growth factor receptor (FGFR) 1, FGFR2, FGFR3, CD 90, and vimentin.

Fibroblasts may be used alone or as a mixture of two or more. The fibroblasts are not particularly limited, and, for example, they may be derived from human, monkey, dog, cat, rabbit, pig, cow, or rat. Among them, the fibroblasts are preferably derived from human.

The cell cluster may further include vascular endothelial cells in addition to the fibroblasts. The vascular endothelial cells are not particularly limited, and, for example, they may be derived from human, monkey, dog, cat, rabbit, pig, cow, mouse, or rat. Among them, the vascular endothelial cells are preferably derived from human.

Whether a cell is a vascular endothelial cell can be determined from the morphology of the cell observed under a microscope or by the expression of marker molecules from the cell. Markers indicating vascular endothelial cells include CD 31, VEGFR-2, and Tie-2/Tek.

The cell cluster may include cells other than fibroblasts and vascular endothelial cells. Such cells include, for example, somatic cells, germ cells, induced pluripotent stem cells (iPS cells), embryonic stem cells (ES cells), tissue stem cells, and cancer cells derived from bone, muscle, internal organs, nerves, brain, bone, skin, and blood. Examples of cancer cells include those derived from colon cancer, lung cancer, stomach cancer, esophageal cancer, colonic cancer, rectal cancer, pancreatic cancer, breast cancer, ovarian cancer, prostate cancer, renal cell cancer, and liver cancer.

The cells constituting the cell cluster may be primary cells or cultured cells such as subcultured cells and cell line cells.

In step (A), a cell cluster, an extracellular matrix component, and a polymer electrolyte may be further mixed with a cationic substance, the cell cluster containing a fibroblast. As the cationic substance, any substance having a positive charge can be used as long as it does not adversely affect the growth of cells and the formation of cell aggregates. Examples of cationic substances include, but are not limited to, cationic buffers such as tris-hydrochloric acid, trismaleic acid, bis-tris and HEPES; ethanolamine, diethanolamine, triethanolamine, polyvinylamine, polyallylamine, polylysine, polyhistidine, and polyarginine. Among them, a cationic buffer is particularly preferable, and tris-hydrochloric acid is even more preferable.

The concentration of the cationic substance is not particularly limited as long as they do not adversely affect the growth of cells and the formation of cell aggregates. The concentration of the cationic substance used in the present embodiment may preferably be 10 to 100 mM, and may be, for example, 20 to 90 mM, may be, for example, 30 to 80 mM, and may be, for example, 40 to 70 mM, and may be, for example 45 to 60 mM.

In the case where a cationic buffer is used as the cationic substance, the pH of the cationic buffer solution is not particularly limited as long as it does not adversely affect the growth of cells and the formation of cell aggregates. The pH of the cationic buffer solution used in the present embodiment is preferably 6.0 to 8.0. For example, the pH of the cationic buffer solution used in the present embodiment may be 7.0, 7.1, 7.2, 7.3, 7.4, 7.5, 7.6, 7.7, 7.8, 7.9, or 8.0. The pH of the cationic buffer solution used in the present embodiment is more preferably 7.2 to 7.6, and 7.4 is even more preferable.

As the extracellular matrix component, any component constituting the extracellular matrix (ECM) can be used as long as the component does not adversely affect the growth of cells and the formation of cell aggregates. Examples of the extracellular matrix component include, but are not limited to, collagen, laminin, fibronectin, vitronectin, elastin, tenascin, entactin, fibrillin, proteoglycan, and modifications or variants thereof. The extracellular matrix component may be used singly or in combination of two or more kinds.

Examples of the proteoglycan include chondroitin sulfate proteoglycan, heparan sulfate proteoglycan, keratan sulfate proteoglycan, and dermatan sulfate proteoglycan. As the extracellular matrix component, among others, collagen, laminin and fibronectin are preferable, and collagen is particularly preferable.

Concentrations of extracellular matrix components are not particularly limited and are preferably more than 0 mg/mL and less than 1.0 mg/mL, as long as they do not adversely affect cell growth and the formation of cell aggregates. The concentration of the extracellular matrix component may preferably be 0.005 mg/mL or more and 1.0 mg/mL or less, 0.01 mg/mL or more to 1.0 mg/mL or less, 0.025 mg/mL or more and 1.0 mg/mL or less, and 0.025 mg/mL or more and 0.1 mg/mL or less. The extracellular matrix component may be used by dissolving it in an appropriate solvent. Examples of solvents include, but are not limited to, water, buffer solutions, and aqueous acetic acid solutions. Among them, buffer solutions or aqueous acetic acid solutions is preferred.

The term “polymer electrolyte” as used in the present specification refers to a polymer having a dissociable functional group in the polymer chain. As the polymer electrolyte used in the present embodiment, any polymer electrolyte can be used as long as it does not adversely affect the growth of cells and the formation of cell aggregates. Examples of the polymer electrolyte include, but are not limited to, glycosaminoglycans such as heparin, chondroitin sulfate (e.g., chondroitin 4-sulfate and chondroitin 6-sulfate), heparan sulfate, dermatan sulfate, keratan sulfate, hyaluronic acid, and the like; dextran sulfate, rhamnan sulfate, fucoidan, carrageenan, polystyrene sulfonic acid, polyacrylamide-2-methylpropanesulfonic acid, polyacrylic acid, and derivatives thereof. Any one of these polymer electrolytes may be used singly or in combination of two or more kinds.

The polymer electrolyte used in the present embodiment may preferably be a glycosaminoglycan. Among them, heparin, chondroitin sulfate, and dermatan sulfate are preferred, and heparin is particularly preferred.

The concentration of the polymer electrolyte in the production method of the present embodiment is not particularly limited as long as they do not adversely affect the growth of cells and the formation of cell aggregates. The concentration of the polymer electrolyte may preferably be more than 0 mg/mL and less than 1.0 mg/mL, may be 0.005 mg/mL or more and 1.0 mg/mL or less, may be 0.01 mg/mL or more and 1.0 mg/mL or less, may be 0.025 mg/mL or more and 1.0 mg/mL or less, and may be 0.025 mg/mL or more and 0.1 mg/mL or less.

The polymer electrolyte may be used by dissolving it in an appropriate solvent. Examples of the solvents include, but are not limited to, water and buffer solutions. When a cationic buffer solution is used as the cationic substance, a polymer electrolyte may be dissolved in the cationic buffer solution.

The blending ratio (weight ratio) of the polymer electrolyte to the extracellular matrix component may preferably be 1:2 to 2:1, may be 1:1.5 to 1.5:1, or may be 1:1.

In step (A), the mixing of a cell cluster, an extracellular matrix component, and a polymer electrolyte can be carried out in a suitable container such as a dish, tube, flask, bottle, or plate. The cell cluster contains a fibroblast. These mixtures may be performed in containers used in the following step (B).

Subsequently, in step (B), a cell aggregate is obtained from the mixture obtained in step (a). The term “cell aggregate” as used in the present specification refers to a structure in which cells are assembled into a single unit. The cell aggregate also includes a cell deposit obtained by centrifugation, filtration, or the like. In an embodiment, the cell aggregate is a slurry-like viscous body. The term “slurry-like viscous body” refers to a gel-like cell aggregate like the one described in Akihiro Nishiguchi et al., “Cell-cell crosslinking by bio-molecular recognition of heparin-based layer-by-layer nanofilms”, Macromol Biosci., 15 (3), 312-317, 2015.

Cell aggregates may be formed by placing the mixture obtained by the above steps in a suitable container and allowing it to stand. Alternatively, cell aggregates may be formed by placing the mixture obtained by the above steps in a suitable container and collecting the cells by, for example, centrifugation, magnetic separation or filtration. When cells are collected by centrifugation, magnetic separation, or filtration, the liquid portion may or may not be removed.

The vessel used in step (B) may be a culture vessel used to culture cells. The culture vessel may be a vessel having a material and a shape normally used in cell or microorganism culture. Examples of the material of the culture vessel include, but are not limited to, glass, stainless steel, and plastic. Examples of culture vessels include, but are not limited to, a dish, tube, flask, bottle, and plate. The container is preferably formed, at least in part, of a material that does not allow cells in the liquid to pass through, but allows the liquid to pass through. Examples of such vessels include, but are not limited to, cell culture inserts such as Transwell (registered trademark) inserts, Netwell (registered trademark) inserts, Falcon (registered trademark) cell culture inserts, and Millicell (registered trademark) cell culture inserts.

The conditions of centrifugation are not particularly limited as long as they do not adversely affect the growth of cells. For example, the cells may be collected by seeding the mixture in a cell culture insert and centrifuging it at 400 × g at 10° C. for one minute.

Subsequently, in step (C), the cell aggregate is cultured to obtain three-dimensional cell tissue. When the cell aggregates are cultured, adhesion between cells of the cell aggregates is promoted, resulting in a stable three-dimensional cell tissue. Culturing cell aggregates for 5 minutes to 72 hours is thought to yield three-dimensional cell tissue.

The culture of cell aggregates can be carried out under culture conditions suitable for the cells to be cultured. Those skilled in the art can select an appropriate medium according to the cell type and desired function. The medium is not particularly limited but includes, for example, D-MEM, E-MEM, MEMα, RPMI-1640, McCoy’s 5A, Ham’s F-12, and the like, and a medium to which serum is added at about 1 to 20% by volume. Examples of serum include bovine serum (CS), fetal bovine serum (FBS), and fetal horse serum (HBS). Conditions such as temperature and atmospheric composition of the culture environment may be adjusted to the conditions suitable for the cells to be cultured.

The cell aggregate may be suspended in a solution prior to culturing. The solution is not particularly limited as long as it does not adversely affect the growth of cells and the formation of three-dimensional cell tissue. For example, a medium, a buffer solution, or the like suitable for the cells constituting the cell aggregate can be used. The suspension of the cell aggregate can be carried out in a suitable container such as a dish, tube, flask, bottle, or plate.

When the cell aggregate is suspended in a solution, it is also possible to precipitate the cells and form a cell deposit before culturing. Precipitation of cells can be performed, for example, by centrifugation. The conditions of centrifugation are not particularly limited as long as they do not adversely affect the growth of cells and the formation of cell aggregates. For example, a suspension of cell aggregates may be precipitated by centrifugation at 400 to 1,000 × g for 1 minute at room temperature. Alternatively, the cells may be precipitated by spontaneous sedimentation.

The container used in the step (C) may be the same as the container used in the step (B). In step (C), the container used in step (B) may be used as it is or transferred to another container.

Upon cell culture, a substance for suppressing deformation of the constructed three-dimensional cell tissue (for example, tissue contraction, peeling at the tissue edge, or the like) may be added to the medium. An example of such a substance is Y-27632, which is Rhoassociated coiled-coil forming kinase/Rho-binding kinase (ROCK) inhibitor, but the substance is not limited to this.

Step (C) may be performed after steps (A) and (B) are performed two or more times. By repeating steps (A) and (B), cell aggregates or cell deposits can be laminated to produce three-dimensional cell tissue having a plurality of layers. That is, three-dimensional cell tissue having a large thickness can be produced.

In addition, when steps (A) and (B) are repeated to laminate cell aggregates or cell deposits, a different cell cluster may be used for each repetition to laminate three-dimensional cell tissues formed of different types of cells. For example, after the first step (A) and step (B) are performed, the second step (A) is performed using a different cell cluster from the first step (A). Then, by performing the second step (B), a layer containing the cell cluster used in the second step (A) can be formed on the layer containing the cell cluster used in the first step (A). Multiple repetition of the steps (A) and (B) leads to a lamination of three-dimensional cell tissue composed of a plurality of types of cell clusters.

In the method for producing the conditioned medium of the present embodiment, the thickness of the three-dimensional cell tissue is preferably 5 µm or more. The term “thickness of three-dimensional cell tissue” as used in the present specification refers to the maximum thickness of a section of the three-dimensional cell tissue taken along a line passing through the center of gravity when viewed from the top of the three-dimensional cell tissue. Here, the thickness of the section of the three-dimensional cell tissue taken along the line passing through the center of gravity when viewed from the top of the three-dimensional cell tissue is the thickness of the section in approximately the center of the three-dimensional cell tissue. The thickness of the section of the three-dimensional cell tissue can also be referred to as the maximum thickness of the three-dimensional cell tissue measured in a section obtained by cutting the three-dimensional cell tissue along a line that passes through the center of gravity when the three-dimensional cell tissue is viewed from the top. The shape of the three-dimensional cell tissue depends on the container used to produce the three-dimensional cell tissue, but when the three-dimensional cell tissue is produced using, for example, a cylindricalshaped cell culture insert, it becomes a cylindrical shape. In this case, the shape of the three-dimensional cell tissue when viewed from the top is a circle, and the center of gravity when viewed from the top is the center of the circle. The shape of the three-dimensional cell tissue is not limited to a cylindrical shape and can be any shape according to the purpose. Specifically, for example, a polygonal prism shape such as a triangular prism shape and a quadrangular prism shape can be exemplified.

Three-dimensional cell tissue with a thickness of 5 µm or more tends to ensure a sufficient number of cells in the cell cluster containing fibroblasts, and to make it easier to obtain conditioned medium suitable for use in culturing patient-derived cancer cells. The upper limit of the thickness of the three-dimensional cell tissue is not particularly limited, but 300 µm or less is realistic.

In the method of producing the conditioned medium of the present embodiment, the three-dimensional cell tissue described above is cultured in the first medium for 24 hours or longer. The first medium is not particularly limited but may be other cell culture media such as Ham’s F-10 Nutrient Mixture, Ham’s F-12 Nutrient Mixture, and RPMI 1640 Media, in addition to Dulbecco’s Modified Eagle Medium (DMEM). The composition of a typical DMEM medium is shown in Table 1 below.

TABLE 1

| Component | Concentration (mg/L) |
|---|---|
| Glycine | 30 |
| L-Arginine HCl | 84 |
| L-Cysteine 2HCl | 63 |
| L-Histidine HCl H₂O | 42 |
| L-Isoleucine | 105 |
| L-Leucine | 105 |
| L-Lysine HCl | 146 |
| L-Methionine | 30 |
| L- Phenylalanine | 66 |
| L-Serine | 42 |
| L-Threonine | 95 |
| L- Tryptophan | 16 |
| L- Tyrosin 2Na 2H₂O | 104 |
| L-Valine | 94 |
| Choline chloride | 4 |
| D-Calcium pantothenate | 4 |
| Folic acid | 4 |
| Nicotinamide | 4 |
| Pyridoxal hydrochloride | 4 |
| Riboflavin | 0.4 |
| Thiamine hydrochloride | 4 |
| i-Inositol | 7.2 |
| Iron(III) nitrate nonahydrate | 0.1 |
| MgSO₄ | 97.67 |
| KCl | 400 |
| NaHCO₃ | 3700 |
| NaCl | 6400 |
| NaH₂PO₄ H₂O | 125 |
| D-Glucose | 4500 |
| Phenol red | 15 |

The incubation time of the three-dimensional cell tissue in the first medium is the appropriate time for a sufficient amount of the active ingredient to be secreted from the three-dimensional cell tissue. The upper limit of the culture time of the three-dimensional cell tissue in the first medium is not particularly limited, but approximately 24 to 96 hours is realistic. Culturing for 12 hours or less makes it difficult to secrete sufficient amounts of active ingredients. Culturing for 120 hours or more would tend to decrease the survival rate of the cells in the three-dimensional cell tissue and would make it difficult to increase the amount of active ingredients in the first medium. That is, the culture time of the three-dimensional cell tissue in the first medium may be 12 hours or more and 120 hours or less, 24 hours or more and 96 hours or less, and 24 hours or more and 48 hours or less.

### Conditioned Medium

In one embodiment, the present invention provides a conditioned medium for culturing a patient-derived cancer cell, which is produced by the production method described above. As will be described later in the Examples, by using the conditioned medium of the present embodiment, patient-derived cancer cells can be cultured without using a special medium. The factors included in this conditioned medium of the present embodiment that enable the culture of patient-derived cancer cells have not been identified.

### Method of Culturing a Patient-Derived Cancer Cell

According to one embodiment, the present invention provides a method of culturing a patient-derived cancer cell, the method including: culturing a patient-derived cancer cell in a second medium containing a conditioned medium for culturing a patient-derived cancer cell, wherein the conditioned medium is made by culturing three-dimensional cell tissue in a first medium for 24 hours or longer, the three-dimensional cell tissue containing a cell cluster, an extracellular matrix component, and a polymer electrolyte, the cell cluster containing a fibroblast, and collecting the first medium in which the three-dimensional cell tissue has been cultured for use as the conditioned medium for culturing a patient-derived cancer cell.

In the method of the present embodiment, the conditioned medium for culturing a patient-derived cancer cell is the same as that described above. As will be described later in the Examples, the method of the present embodiment enables patient-derived cancer cells to be cultured without using a special medium.

In the method of the present embodiment, the second medium may be formed of only conditioned medium for culturing patient-derived cancer cells, or it may be a medium in which conditioned medium for culturing patient-derived cancer cells is diluted with other medium. Here, other media may be the same media used for the first medium, such as DMEM media, Ham’s F-10 Nutrient Mixture, Ham’s F-12 Nutrient Mixture, and RPMI 1640 Media. Other media may contain additives such as serum and antibiotics.

The ratio of conditioned medium for culturing a patient-derived cancer cell in the second medium is preferably 20% or more by volume of the total volume of the second medium. The ratio of conditioned medium for culturing a patient-derived cancer cell in the second medium is preferably 40% or more by volume of the total volume of the second medium. As described later in the Examples, when the ratio of conditioned medium for culturing a patient-derived cancer cell in the second medium is within the above range, a patient-derived cancer cell can be cultured better than when using a normal medium such as DMEM medium.

### EXAMPLES

While the present invention will be described in more detail using the following Examples, it should be understood that the present invention is not limited to the following Examples.

### Production Example 1

### Preparation of Conditioned Medium of Production Example 1

2 × 10⁷ NHDF (model number “CC-2509”, LONZAKK.), which are human neonatalderived skin fibroblasts, were suspended in 50 mM tris-hydrochloric acid buffer solution (pH 7.4) containing 0.05 mg/mL heparin and 0.05 mg/mL collagen. Collagen I was used as the collagen.

Subsequently, the cell suspension was centrifuged at 1000 × g for 1 minute at room temperature, the supernatant was removed and resuspended in an appropriate amount of DMEM medium containing 10% fetal bovine serum (FBS). The cell suspension was then seeded into 100 mm Transwell culture inserts (model number “3419”, Corning Incorporated) to obtain three-dimensional cell tissues. The thickness of the three-dimensional cell tissue was approximately 100 µm.

Subsequently, an appropriate amount of 10% FBS-DMEM was added and incubated in a CO₂ incubator (37° C., 5% CO₂) for 96 hours or more. The culture supernatant was then collected from the 100 mm Transwell culture insert to obtain the conditioned medium of Production Example 1.

### Production Example 2

### Preparation of Conditioned Medium of Production Example 2

2 × 10⁷ NHDF (model number “CC-2509”, LONZAKK.), which are human neonatalderived skin fibroblasts, and 3 × 10⁵ HUVEC (model number “”CC-2517A”, LONZA KK.), which are human umbilical vein endothelial cells, were suspended in 50 mM tris-hydrochloric acid buffer solution (pH 7.4) containing 0.05 mg/mL heparin and 0.05 mg/mL collagen. Collagen I was used as the collagen.

Subsequently, the cell suspension was centrifuged at 1000 × g for 1 minute at room temperature, the supernatant was removed and resuspended in an appropriate amount of DMEM medium containing 10% fetal bovine serum (FBS). The cell suspension was then seeded into 100 mm Transwell culture inserts (model number “3419”, Corning Incorporated) to obtain three-dimensional cell tissues. The thickness of the three-dimensional cell tissue was approximately 100 µm.

Subsequently, an appropriate amount of 10% FBS-DMEM was added and incubated in a CO₂ incubator (37° C., 5% CO₂) for 96 hours or more. The culture supernatant was then collected from the 100 mm Transwell culture insert to obtain the conditioned medium of Production Example 2.

### Production Example 3

### Preparation of Conditioned Medium of Production Example 3

2 × 10⁷ NHDF (model number “CC-2509″, LONZAKK.), which are human neonatalderived skin fibroblasts, and 3 × 10⁵ HUVEC (model number “’’CC-2517A”, LONZA KK.), which are human umbilical vein endothelial cells, were suspended in 50 mM tris-hydrochloric acid buffer solution (pH 7.4) containing 0.05 mg/mL heparin and 0.05 mg/mL collagen. Collagen I was used as the collagen.

Subsequently, the cell suspension was centrifuged at 1000 × g for 1 minute at room temperature, the supernatant was removed and resuspended in an appropriate amount of DMEM medium containing 10% fetal bovine serum (FBS). The cell suspension was then seeded into 100 mm Transwell culture inserts (model number “3419”, Corning Incorporated) to obtain three-dimensional cell tissues. The thickness of the three-dimensional cell tissue was approximately 100 µm.

Subsequently, an appropriate amount of 10% FBS-DMEM was added and incubated in a CO₂ incubator (37° C., 5% CO₂) for 24 hours. The supernatant was then removed and 1.5 × 10⁶ cells patient-derived cancer cells, PDC-Colon (colon-derived, model number “CE-HC-105”, Eolas Biosciences Co. Ltd.) were seeded. The medium used was 10% FBS-DMEM. They were then incubated in a CO₂ incubator (37° C., 5% CO₂) for 96 hours or more. The culture supernatant was then collected from the 100 mm Transwell culture insert to obtain the conditioned medium of Production Example 3.

### Experimental Example 1

Patient derived cancer cells were cultured using the conditioned medium of Production Examples 1 to 3. As the patient-derived cancer cells, PDC-Colon (colon-derived, model number “CE-HC-105”, Eolas Biosciences Co. Ltd.) was used.

As the medium, the conditioned medium of Production Examples 1 to 3 and a medium obtained by mixing these conditioned mediums with 10% FBS-DMEM were used. The mixing ratio of the conditioned medium and 10% FBS-DMEM was 75:25 (75% of the conditioned medium in the medium) or 50:50 (50% of the conditioned medium in the medium) by volume. For comparison, as a medium, a group using 10% FBS-DMEM and a group using a special medium (ES cell culture medium, Gibco) were also prepared.

Specifically, first, PDC-Colons were suspended in each medium and seeded in wells of a 96-well plate at 4× 10³ cells per well. They were then incubated in a CO₂ incubator (37° C., 5% CO₂) for 7 days.

Subsequently, cells were collected by trypsinization, stained with trypan blue, and the number of living cells was measured using a cell counting device (product name: “Countess II FL”, Thermo Fisher Scientific K.K.). Survival rate was 90% or more for both groups.

The following Table 2 shows the measurement results of the number of living cells per well for each group of patient-derived cancer cells. In Table 2, “Production Example 1” means the conditioned medium of Production Example 1, “Production Example 2” means the conditioned medium of Production Example 2, and “Production Example 3” means the conditioned medium of Production Example 3.

The results showed that the number of patient-derived cancer cells when conditioned medium of Production Examples 1 to 3 was used was higher than that when the cells were cultured in 10% FBS-DMEM, regardless of the ratio of conditioned medium in the medium.

The results showed that the number of patient-derived cancer cells when conditioned medium of Production Examples 1 to 3 was used was equal to or greater than that when the cells were cultured in a special medium, regardless of the ratio of conditioned medium in the medium.

This result shows that cells collected from a living organism can be cultured by using the conditioned medium of Production Examples 1 to 3.

TABLE 2

| Medium | Number of living cells (cell) |
|---|---|
| 10% FBS-DMEM | 3,800±2,150 |
| Special medium | 27,367±1,605 |
| Production Example 1 | 28,267±1,470 |
| Production Example 1: 10% FBS-DMEM = 75:25 | 32,267±2,089 |
| Production Example 1: 10% FBS-DMEM = 50:50 | 31,167±3,565 |
| Production Example 2 | 35,833±1,396 |
| Production Example 2: 10% FBS-DMEM = 75:25 | 38,900±5,294 |
| Production Example 2: 10% FBS-DMEM = 50:50 | 41,133±5,514 |
| Production Example 3 | 36,033±3,227 |
| Production Example 3: 10% FBS-DMEM = 75:25 | 37,867±205 |
| Production Example 3: 10% FBS-DMEM = 50:50 | 35,267±3,535 |

### Experimental Example 2

### Culture 2 of Patient-Derived Cancer Cells

Patient derived cancer cells were cultured using the conditioned medium of Production Example 1. As the patient-derived cancer cells, PDC-Colon (colon-derived, model number “CE-HC-105”, Eolas Biosciences Co. Ltd.) was used.

As the medium, the conditioned medium of Production Example 1 and a medium obtained by mixing the conditioned medium of Example 1 with 10% FBS-DMEM were used. The mixing ratio of conditioned medium and 10% FB S-DMEM was 75:25 (75% of conditioned medium in the medium), 50:50 (50% of conditioned medium in the medium), 40:60 (40% of conditioned medium in the medium), 30:70 (30% of conditioned medium in the medium), 20:80 (20% of conditioned medium in the medium), and 10:90 (10% of conditioned medium in the medium) by volume. For comparison, as a medium, a group using 10% FBS-DMEM and a group using a special medium (ES cell culture medium, Gibco) were also prepared.

Specifically, first, PDC-Colons were suspended in each medium and seeded in wells of a 96-well plate at 4× 10³ cells per well. They were then incubated in a CO₂ incubator (37° C., 5% CO₂) for 7 days.

Subsequently, cells were collected by trypsinization, stained with trypan blue, and the number of living cells was measured using a cell counting device (product name: “Countess II FL”, Thermo Fisher Scientific K.K.). Survival rate was 90% or more for both groups.

The following Table 3 shows the measurement results of the number of living cells per well for each group of patient-derived cancer cells. In Table 3, “Production Example 1” means the conditioned medium of Production Example 1. As a result, the number of patient-derived cancer cells was higher when cultured in a medium containing 20% or more by volume of conditioned medium of Production Example 1, compared to when cultured in 10% FBS-DMEM.

In addition, the number of patient-derived cancer cells was higher when cultured in a medium containing 50% or more by volume of conditioned medium of Production Example 1, compared to when cultured in a special medium.

TABLE 3

| Medium | Number of living cells (cell) |
|---|---|
| 10% FBS-DMEM | 3,800±2,150 |
| Special medium | 27,367±1,605 |
| Production Example 1 | 28,267±1,470 |
| Production Example 1: 10% FBS-DMEM = 75:25 | 32,267±2,089 |
| Production Example 1: 10% FBS-DMEM = 50:50 | 31,167±3,565 |
| Production Example 1: 10% FBS-DMEM = 40:60 | 23,367±2,705 |
| Production Example 1: 10% FBS-DMEM = 30:70 | 9,477±2,720 |
| Production Example 1: 10% FBS-DMEM = 20:80 | 7,553±2,720 |
| Production Example 1: 10% FBS-DMEM = 10:90 | 3,397±1,579 |

### Experimental Example 3

### Culture 3 of Patient-Derived Cancer Cells

Patient derived cancer cells were cultured using the conditioned medium of Production Example 2. As the patient-derived cancer cells, PDC-Lung (lung-derived, model number “LF-HC-088”, Eolas Biosciences Co. Ltd.) was used.

As the medium, the conditioned medium of Production Example 2 and a medium obtained by mixing the conditioned medium of Example 2 with 10% FBS-DMEM were used. The mixing ratio of conditioned medium and 10% FB S-DMEM was 75:25 (75% of conditioned medium in the medium), 50:50 (50% of conditioned medium in the medium), 40:60 (40% of conditioned medium in the medium), 30:70 (30% of conditioned medium in the medium), 20:80 (20% of conditioned medium in the medium), and 10:90 (10% of conditioned medium in the medium) by volume. For comparison, as a medium, a group using 10% FBS-DMEM and a group using a special medium (ES cell culture medium, Gibco) were also prepared.

Specifically, first, PDC-Lungs were suspended in each medium and seeded in wells of a 96-well plate at 4×10³ cells per well. They were then incubated in a CO₂ incubator (37° C., 5% CO₂) for 7 days.

Subsequently, cells were collected by trypsinization, stained with trypan blue, and the number of living cells was measured using a cell counting device (product name: “Countess II FL”, Thermo Fisher Scientific K.K.). Survival rate was 90% or more for both groups.

The following Table 4 shows the measurement results of the number of living cells per well for each group of patient-derived cancer cells. In Table 4, “Production Example 2” means the conditioned medium of Production Example 2. As a result, the number of patient-derived cancer cells was higher when cultured in a medium containing 10% or more by volume of conditioned medium of Production Example 2, compared to when cultured in 10% FBS-DMEM.

In addition, the number of patient-derived cancer cells was higher when cultured in a medium containing 40% or more by volume of conditioned medium of Production Example 1, compared to when cultured in a special medium.

TABLE 4

| Medium | Number of living cells (cell) |
|---|---|
| 10% FBS-DMEM | 12,810±6,515 |
| Special medium | 49,567±2,053 |
| Production Example 2 | 50,867±7,587 |
| Production Example 2: 10% FBS-DMEM = 75:25 | 53,800±7,360 |
| Production Example 2: 10% FBS-DMEM = 50:50 | 61,300±2,491 |
| Production Example 2: 10% FBS-DMEM = 40:60 | 50,467±1,948 |
| Production Example 2: 10% FBS-DMEM = 30:70 | 43,733±8,192 |
| Production Example 2: 10% FBS-DMEM = 20:80 | 26,000±5,813 |
| Production Example 2: 10% FBS-DMEM = 10:90 | 14,733±4,714 |

An aspect of the present invention is to provide a technique for culturing cells taken from living organisms.

The present invention has the following aspects.

A method of producing a conditioned medium for culturing a patient-derived cancer cell, the method including: culturing three-dimensional cell tissue in a first medium for 24 hours or longer, the three-dimensional cell tissue containing a cell cluster, an extracellular matrix component, and a polymer electrolyte, the cell cluster containing a fibroblast; and collecting the first medium in which the three-dimensional cell tissue has been cultured for use as the conditioned medium for culturing a patient-derived cancer cell.

The method of producing a conditioned medium for culturing a patient-derived cancer cell according to [1], wherein the three-dimensional cell tissue has a thickness of 5 µm or more.

A method of producing a conditioned medium for culturing a patient-derived cancer cell according to [1] or [2], wherein the cell cluster further comprises a vascular endothelial cell.

The method of producing a conditioned medium for culturing a patient-derived cancer cell according to any one of [1] to [3], wherein the first medium is DMEM medium.

A conditioned medium for culturing a patient-derived cancer cell, wherein the conditioned medium is produced by the method according to any one of [1] to [4].

A method of culturing a patient-derived cancer cell, the method including: culturing a patient-derived cancer cell in a second medium containing a conditioned medium for culturing a patient-derived cancer cell, wherein the conditioned medium is made by culturing three-dimensional cell tissue in a first medium for 24 hours or longer, the three-dimensional cell tissue containing a cell cluster, an extracellular matrix component, and a polymer electrolyte, the cell cluster containing a fibroblast, and collecting the first medium in which the three-dimensional cell tissue has been cultured for use as the conditioned medium for culturing a patient-derived cancer cell.

The method of culturing a patient-derived cancer cell according to [6], wherein the three-dimensional cell tissue has a thickness of 5 µm or more.

The method of culturing a patient-derived cancer cell according to [6] or [7], wherein the cell cluster further comprises a vascular endothelial cell.

The method of culturing a patient-derived cancer cell according to any one of [6] to [8], wherein the first medium is DMEM medium.

The method of culturing a patient-derived cancer cell according to any one of [6] to [9], wherein a ratio of the conditioned medium for culturing a patient-derived cancer cell in the second medium is 20% or more by volume.

The method of culturing a patient-derived cancer cell according to any one of [6] to [10], wherein the second medium contains DMEM medium.

### Industrial Applicability

The present application provides a technique for culturing cells taken from living organisms.

Obviously, numerous modifications and variations of the present invention are possible in light of the above teachings. It is therefore to be understood that within the scope of the appended claims, the invention may be practiced otherwise than as specifically described herein.

What is claimed is:

1. A method of producing a conditioned medium for culturing a patient-derived cancer cell, the method comprising:
culturing, in a first medium for 24 hours or longer, a three-dimensional cell tissue including an extracellular matrix component, a polymer electrolyte, and a cell cluster including a fibroblast; and
collecting the first medium in which the three-dimensional cell tissue has been cultured as the conditioned medium.

2. The method according to claim 1, wherein the three-dimensional cell tissue has a thickness of 5 µm or more.

3. The method according to claim 1, wherein the cell cluster further comprises a vascular endothelial cell.

4. The method according to claim 1, wherein the first medium is DMEM medium.

5. A conditioned medium for culturing a patient-derived cancer cell, produced by a process including the method of claim 1.

6. A method of culturing a patient-derived cancer cell, comprising:
preparing a conditioned medium; and
culturing a patient-derived cancer cell in a second medium including the conditioned medium for culturing a patient-derived cancer cell,
wherein the preparing includes
culturing, in a first medium for 24 hours or longer, a three-dimensional cell tissue including an extracellular matrix component, a polymer electrolyte, and a cell cluster including a fibroblast, and
collecting the first medium in which the three-dimensional cell tissue has been cultured as the conditioned medium.

7. The method according to claim 6, wherein the three-dimensional cell tissue has a thickness of 5 µm or more.

8. The method according to claim 6, wherein the cell cluster further comprises a vascular endothelial cell.

9. The method according to claim 6, wherein the first medium is DMEM medium.

10. The method according to claim 6, wherein the second medium includes the conditioned medium at a ratio of 20% or more by volume.

11. The method according to claim 6, wherein the second medium contains DMEM medium.
